(19) United States
(12) Patent Application Publication
(10) Pub. No.: US 2014/0120498 A1
(43) Pub. Date: May 1, 2014

(54) DENTAL IMPLANT AND DENTAL IMPLANT SYSTEM

(71) Applicant: DENTAURUM GMBH & CO. KG, Ispringen (DE)

(72) Inventors: Tobias GROSSE, Ispringen (DE); Andreas BAUER, Bruchsal (DE); Friedhelm HEINEMANN, Reichshof (DE)

(73) Assignee: DENTAURUM GMBH & CO. KG, Ispringen (DE)

(21) Appl. No.: 14/061,967

(22) Filed: Oct. 24, 2013

(30) Foreign Application Priority Data
Oct. 29, 2012 (DE) 10 2012 110 318.6

(51) Int. Cl. A61C 8/00; A61K 6/04

(52) U.S. Cl. CPC A61C 8/0069; A61C 8/0022; A61C 8/0012; A61K 6/04; A61C 8/0068
USPC 433/174; 433/173

(57) ABSTRACT

A dental implant with a receiver for dental mounting parts is disclosed. The receiver comprises at least one cylindrical wall portion and the dental implant has a support face perpendicular to the longitudinal axis, so that a corresponding first mounting part can be radially supported on the at least one cylindrical wall portion and axially on the support face. The receiver furthermore comprises at least one truncated-cone-shaped wall portion, on which a corresponding second mounting part can be radially and axially supported. A dental implant system is also disclosed, comprising the dental implant and at least one first and at least one second dental mounting part, which can selectively be connected to the dental implant.

### CROSS REFERENCE TO RELATED APPLICATIONS

The present disclosure relates to the subject-matter disclosed in German patent application No. 10 2012 110 318.6 of Oct. 29, 2012, which is incorporated herein by reference in its entirety and for all purposes.

### BACKGROUND OF THE INVENTION

The present invention relates to a dental implant with a receiver for dental mounting parts, the receiver extending along a longitudinal axis of the dental implant and it being possible to axially introduce a dental mounting part into the receiver.

The invention furthermore relates to a dental implant system, comprising a dental implant of this type and at least one first and at least one second dental mounting part, which can selectively be connected to the dental implant, the mounting parts in each case comprising a connecting region, which can be axially introduced into the receiver of the dental implant.

In dental implantology, multi-part arrangements are almost always used, which, on the one hand, comprise the actual dental implant that is implanted to permanently remain in the jawbone. The implant has a connecting location for the arrangement of a dental mounting part, the implant typically being combined with a series of various mounting parts in the course of the implantology treatment: thus, for example, during the healing phase after the insertion of the implant, the connecting location can firstly be protected against the penetration of impurities by a closure element. The implant is then generally connected to an impression post, which is used to determine the precise position and orientation of the dental implant during the preparation of a dental impression. The dental technician requires this information to be able to produce a dental prosthesis (for example an end prosthesis) which is ultimately to be connected to the implant with the required precision. The dental prosthesis is connected by means of a dental mounting part called an abutment to the dental implant. Further mounting parts, which can be connected to the dental implant, are, for example, implant holders and gingiva formers.

The connection between the dental implant and the mounting part may, in principle, be configured as an internal connection or as an external connection, the present invention relating to dental implants with an internal connection, in which the mounting part is introduced into a receiver, which is formed within the implant. With this type of connection, two variants are in principle known from the prior art, namely a cylindrical internal connection and a conical internal connection, the two variants each having advantages and drawbacks.

In the case of the cylindrical internal connection, the receiver of the dental implant has a cylindrical wall portion, which is used to receive in shape-locking manner a corresponding cylindrical connecting region of the mounting part. To fix the axial position of the mounting part in relation to the implant, there is used in this case a support face perpendicular to the longitudinal axis, on which the mounting part is supported. The position of the mounting part defined by the support face is very precise, as it is practically not influenced by manufacturing tolerances, which is the significant advantage of the cylindrical internal connection. In particular, this can ensure that the axial position of an impression post precisely corresponds to the later axial position of an abutment, so that any reprocessing in this regard of the dental prosthesis produced by the dental technician is unnecessary. The precisely defined support face also facilitates the practicability by means of CAD/CAM technology. A drawback of the cylindrical internal connection is that when there is a very high transversal introduction of force into the mounting part, a gap can form between the implant and the mounting part as the fit of the mounting part in the cylindrical wall portion of the receiver cannot be made completely free of play.

In the conical internal connection, the receiver of the dental implant has a truncated-cone-shaped wall portion tapering from the coronal end in the apical direction. A corresponding truncated-cone-shaped connecting region of the dental mounting part is introduced into this cone, so both the radial and the axial position of the mounting part are defined by the shape-locking mutual engagement of the corresponding cones. The advantages of this type of internal connection are the good force transmission from the mounting part to the implant, which is linked with a substantially smaller gap formation at high transversal forces, and facilitated handling during the introduction of the narrower apical end of the mounting part into the receiver of the implant. On the other hand, it is disadvantageous that the axial position of the mounting part is influenced to a substantially greater degree by manufacturing tolerances and is therefore less precise than in the cylindrical internal connection. As the conical connection is not only shape-locking, but optionally also frictional, the position of the mounting part also depends on the axial action of force.

Weighing up the respective advantages and drawbacks, the dentist has to make a decision in favour of a system with a cylindrical or a conical internal connection before beginning an implantology treatment and insert a corresponding implant. It is no longer possible to change the connecting system thereafter in the case of the implants according to the prior art. However, this would frequently be desirable, as, for example, in various working steps, a cylindrical connection can sometimes be more advantageous and sometimes a conical connection.

The present invention is based on the object of proposing a dental implant and a dental implant system, which allows the user a greater flexibility in the choice of the connection type between the implant and a dental mounting part.

### SUMMARY OF THE INVENTION

This object is achieved according to the invention in the dental implant of the type mentioned in the introduction in that the receiver comprises at least one cylindrical wall portion and the dental implant has a support face perpendicular to the longitudinal axis, so that a corresponding first mounting part can be supported radially on the at least one cylindrical wall portion and axially on the support face, and the receiver furthermore comprises at least one truncated-cone-shaped wall portion, on which a corresponding second mounting part can be supported radially and axially.

The dental implant according to the invention therefore provides the advantageous possibility of being selectively connected by means of a cylindrical or a conical internal connection to a corresponding dental mounting part. The dentist can thus make a decision in favour of one of the connection types after the insertion of the implant into the jawbone, i.e. he can also take into consideration circumstances in this decision, which have only emerged during or after the implantation. He can, in particular, at any time extend or modify the prosthetic restoration according to the indication, and, depending on this extension, select an optimal connection type (conical or cylindrical). Thus, for example an individual tooth implant with a conical connection of the mounting part, which was set years ago, can be integrated without problems into a milled bar restoration with a cylindrical connection of the mounting part.

The cylindrical and truncated-cone-shaped wall portions are advantageously rotationally symmetrical in each case in relation to the longitudinal axis of the dental implant and arranged consecutively in the axial direction. For example, a cylindrical wall portion can follow a truncated-cone-shaped one or vice versa in the axial direction from the coronal end in the apical direction. The various wall portions do not, however, have to follow one another directly, i.e. a further portion of the receiver, the wall of which is neither cylindrical nor truncated-cone-shaped, may also be arranged between a cylindrical and a truncated-cone-shaped wall portion.

When the dental implant is being connected to the first mounting part, the at least one cylindrical wall portion and the perpendicular support face are used, but not the at least one truncated-cone-shaped wall portion (cylindrical internal connection). When the dental implant is connected to the second mounting part, the at least one truncated-cone-shaped wall portion is used, but not the perpendicular support face (conical internal connection). It is possible, however, during the conical internal connection, for the at least one cylindrical wall portion to also additionally be used for the radial support of the mounting part.

In a preferred embodiment of the invention, the receiver comprises a cylindrical wall portion in the region of its apical end and a truncated-cone-shaped wall portion in the region of its coronal end. The diameter of the receiver in the region of the cylindrical wall portion is, in this case, smaller than the diameter of the receiver in the region of the coronal end of the truncated-cone-shaped wall portion, and preferably also smaller than the diameter in the region of the apical end of the truncated-cone-shaped wall portion. In addition to these two wall portions, the receiver may also comprise further cylindrical and/or truncated-cone-shaped wall portions.

The perpendicular support face preferably extends outwardly from the coronal end of the receiver. The support face may, for example, directly adjoin a truncated-cone-shaped wall portion at the coronal end of the receiver. In this case, the support face is advantageously annular, but it may also have externally, for example, a polygonal border, depending on the configuration of the external contour of the dental implant.

Alternatively, the perpendicular support face may also be formed by an apical base face of the receiver. In this case, it is particularly advantageous if this base face adjoins a cylindrical wall portion at the apical end of the receiver.

In a further advantageous embodiment of the invention, the receiver comprises two cylindrical wall portions, between which a truncated-cone-shaped wall portion is arranged. The two cylindrical wall portions, in this case, have different diameters, the diameter of the apical wall portion naturally being smaller. By providing a plurality of cylindrical wall portions, the stability of the connection between the dental implant and the first dental mounting part can be increased.

Alternatively or additionally, it may be provided that the receiver comprises two truncated-cone-shaped wall portions, between which a cylindrical wall portion is arranged. In this case, it is particularly advantageous if the two truncated-cone-shaped wall portions lie on the same imaginary conical face, as a configuration of this type can be produced relatively easily with a high degree of precision.

The at least one truncated-cone-shaped wall portion preferably has an angle of inclination with respect to the longitudinal axis in the range from about 5° to about 15°. The smaller the angle of inclination, the more readily a force-locking connection can also be achieved to the second dental mounting part in the manner of a self-locking cone, but the axial position of the second mounting part is also all the more dependent on the axial action of force.

It is advantageous if the receiver comprises a portion serving as an anti-rotation device, with which a corresponding portion of a dental mounting part can engage. By means of an anti-rotation device of this type, a defined angular position of the dental mounting part can be defined in relation to the dental implant. The portion serving as an anti-rotation device may be configured in various ways, in particular by recesses distributed along the periphery in the wall of the receiver, in which corresponding projections on the connecting region of the dental mounting part engage.

The portion serving as an anti-rotation device can, in particular, be arranged between a cylindrical and a truncated-cone-shaped wall portion of the receiver. For example, the receiver, from coronal to apical, may comprise a truncated-cone-shaped wall portion, a portion serving as an anti-rotation device and a cylindrical wall portion, the perpendicular support face extending outwardly from the coronal end of the truncated-cone-shaped wall portion.

The mounting part connected to the dental implant is generally fixed by a fastening element, for example a screw, on the dental implant, in particular when the mounting part is an abutment, which is to be permanently and as rigidly as possible connected to the implant. For this purpose, it is preferred if the dental implant has a threaded bore apically adjoining the receiver, into which threaded bore a screw can be screwed to fix the mounting part. Corresponding first and second mounting parts then have an axial bore, through which the screw passes.

The insertion of the dental implant into the jawbone can take place in various ways, which are known per se from the prior art. According to one embodiment of the invention, the dental implant is configured in the form of a bone screw with an external thread for screwing into a jawbone.

The dental implant may be formed from various materials, which are suitable for use in dental implantology. The dental implant is preferably completely or partially made of titanium, a titanium alloy, a cobalt-chromium alloy or ceramic.

In the dental implant system of the type mentioned in the introduction, the object on which the invention is based is achieved according to the invention in that
the connecting region of the at least one first mounting part comprises at least one cylindrical wall portion and a support face perpendicular to the longitudinal axis, so that the corresponding cylindrical wall portions of the receiver and of the connecting region engage one another in shape-locking manner and the corresponding support faces abut one another when the mounting part is connected to the dental implant; and in that
the connecting region of the at least one second mounting part comprises at least one truncated-cone-shaped wall portion, so that the corresponding truncated-cone-shaped wall portions of the receiver and of the connecting region engage one another in shape-locking manner when the mounting part is connected to the dental implant.

The advantages of the implant system according to the invention have already substantially been described in connection with the dental implant according to the invention, the basic idea of the invention being that within this system one and the same dental implant can selectively be connected to a first mounting part by means of a cylindrical internal connection or to a second mounting part by means of a conical internal connection. Advantageously, the implant system comprises a plurality of first mounting parts and a plurality of second mounting parts, the connecting regions of which are in each case configured the same, but which differ with respect to their basic function. The system may thus, for example, comprise an abutment, an impression post etc. with a connecting region for a cylindrical connection as well as an abutment, an impression post etc. with a connection region for a conical connection.

In a preferred embodiment of the invention, the connecting portion of the at least one second mounting part additionally comprises a cylindrical wall portion mutually engaging in shape-locking manner with the corresponding cylindrical wall portion of the receiver when the mounting part is connected to the implant. The cylindrical wall portion of the receiver can thus be used both for the first and the second mounting part for the radial support of the corresponding wall portion of the connecting region, as has already been described above. However, it is important for the difference between the two types of mounting parts that in the at least one second mounting part, no corresponding mating face is present, which could be supported on the support face which is perpendicular to the longitudinal axis of the dental implant.

In a further embodiment of the implant system according to the invention, the connecting region of the at least one first mounting part comprises two cylindrical wall portions and/or the connecting region of the at least one second mounting part comprises two truncated-cone-shaped wall portions. In this case, the receiver of the dental implant also comprises two respectively corresponding wall portions, as has already been described above.

The connecting region of the at least one first mounting part and/or of the at least one second mounting part preferably in each case comprises a portion, which serves as an anti-rotation device, mutually engaging with a corresponding portion of the receiver of the dental implant in shape-locking manner when the mounting part is connected to the dental implant. The anti-rotation device prevents a rotation of the mounting part about the longitudinal axis and allows a precise definition of the angular position in relation to the dental implant. Possible configurations of the anti-rotation device have also already been described in conjunction with the dental implant according to the invention.

### BRIEF DESCRIPTION OF THE DRAWINGS

This and further advantages of the invention will be described in more detail with the aid of the following exemplary embodiment with reference to the Figures, in which, in detail:

FIG. 1 shows a perspective view of a dental implant according to the invention;

FIG. 2 shows a perspective view of a first dental mounting part of a dental implant system according to the invention;

FIG. 3 shows a perspective view of a second dental mounting part of a dental implant system according to the invention;

FIG. 4 shows a view of the dental implant according to FIG. 1 and of the first mounting part according to FIG. 2 in a longitudinal section; and

FIG. 5 shows a view of the dental implant according to FIG. 1 and of the second mounting part according to FIG. 3 in a longitudinal section.

### DETAILED DESCRIPTION OF THE INVENTION

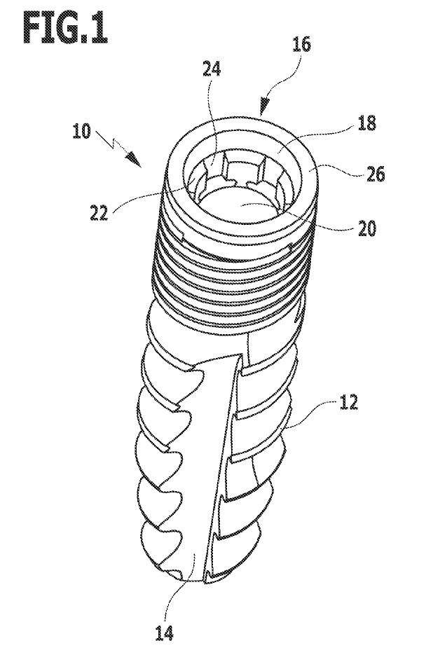

FIG. 1 shows a perspective view of an embodiment of a dental implant according to the invention, which is designated 10 as a whole. The dental implant 10 is configured as a bone screw and has an external thread 12 with a transverse groove 14. The dental implant 10 extends along a longitudinal axis, the coronal end being arranged at the top and the apical end at the bottom in FIG. 1.

The dental implant 10 has a receiver 16, which extends from the coronal end along the longitudinal axis into the dental implant 10. The receiver 16 comprises various wall portions, which are in each case rotationally symmetrical in relation to the longitudinal axis, and specifically in the region of the coronal end, a truncated-cone-shaped wall portion 18 and, in the region of the apical end, a cylindrical wall portion 20. Arranged between these two wall portions is a portion 22, which serves as an anti-rotation device and has recesses 24 arranged along the periphery of the receiver 16.

The dental implant 10 also has a support face 26 perpendicular to the longitudinal axis, which extends outwardly from the coronal end of the receiver 16. The support face 26 is annular.

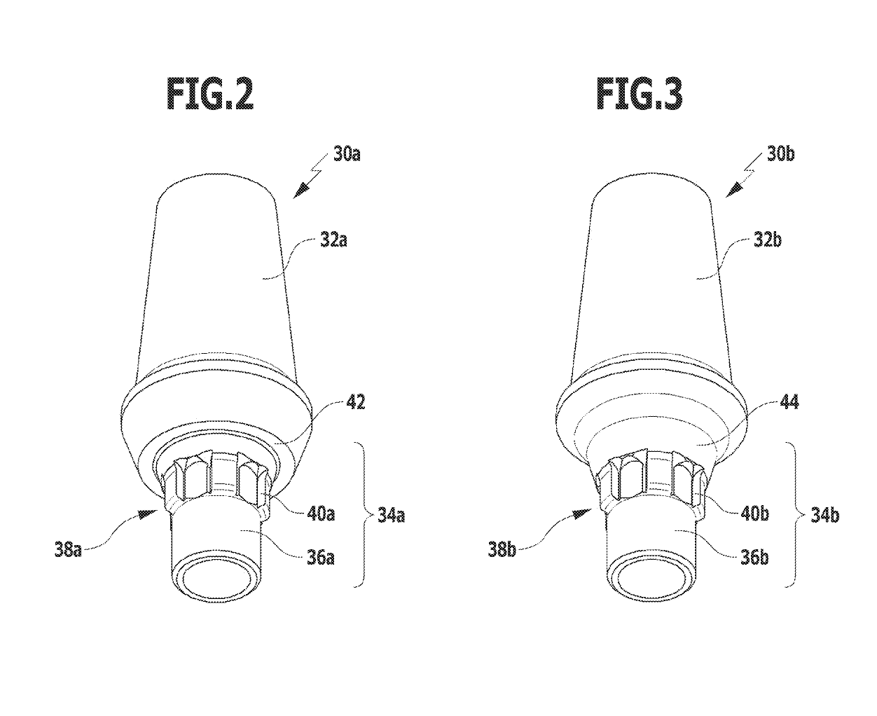

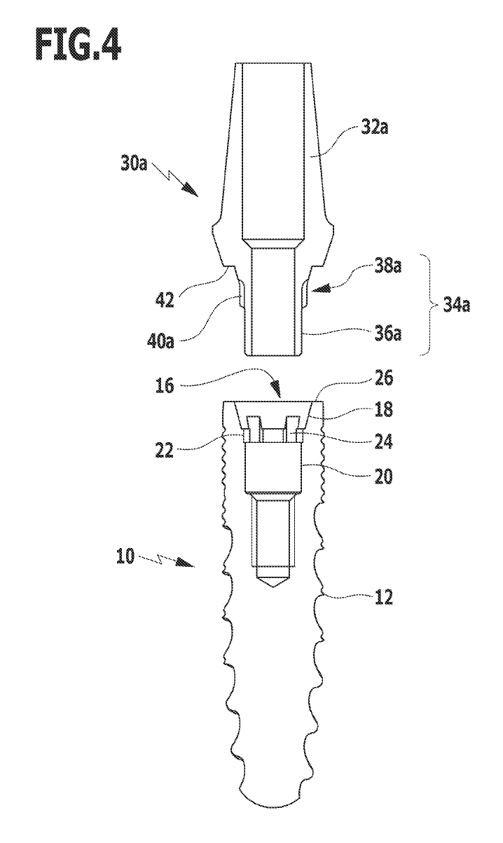

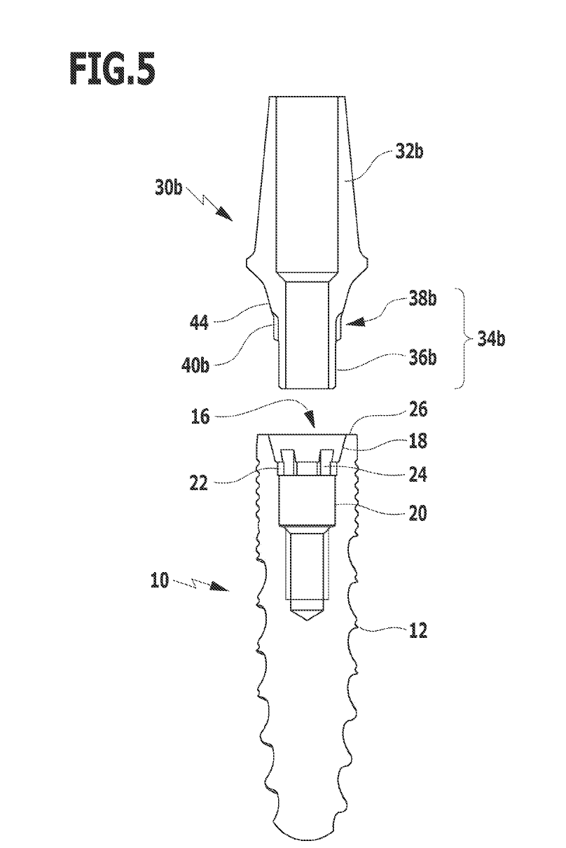

A dental implant system according to the present invention, in addition to the dental implant according to FIG. 1, comprises at least one first dental mounting part, which can be connected by means of a cylindrical connection to the dental implant 10, and at least one second dental mounting part, which can be connected by means of a conical connection to the dental implant 10. An embodiment of a first mounting part 30a is shown perspectively in FIG. 2, and FIG. 4 shows a longitudinal section through the dental implant 10 and the first mounting part 30a. An embodiment of a second mounting part 30b is shown perspectively in FIG. 3, and FIG. 5 shows a longitudinal section through the dental implant 10 and the second mounting part 30b.

The first mounting part 30a is an abutment with a coronal carrier region 32a, on which, for example, an end prosthesis can be applied, and with an apical connecting region 34a to connect the mounting part 30a to the dental implant 10. The connecting region 34a is introduced axially here into the receiver 16.

The connecting region 34a of the first mounting part 30a comprises a cylindrical wall portion 36a at its apical end and a portion 38a adjoining it, which serves as an anti-rotation device. The anti-rotation device 38a comprises projections 40a arranged along the periphery of the connecting region 34a. The connecting region 34a furthermore comprises a support face 42 perpendicular to the longitudinal axis, which is annular and oriented in the apical direction.

When connecting the first mounting part 30a to the dental implant 10, the connecting region 34a is introduced axially into the receiver 16, the corresponding cylindrical wall portions 20 and 36a engaging one another and the projections 40a of the anti-rotation device 38a engaging in the recesses 24 of the anti-rotation device 22. The introduction depth is limited in that the support faces 26 and 42 abut one another. The cylindrical wall portions 20 and 36a are therefore used for radial support and the support faces 26 and 42 for the axial support of the first mounting part 30a. The angular position of the mounting part 30a in relation to the dental implant 10 is defined by the portions 22 and 38a serving as an anti-rotation device.

The second dental mounting part 30b is also an abutment with a carrier region 32b and a connecting region 34b. The connecting region 34b comprises an apical cylindrical wall portion 36b and a portion 38b adjoining it and serving as an anti-rotation device. The portions 36b and 38b of the second mounting part 30b are configured like the corresponding portions 36a and 38a of the first mounting part 30a. At its coronal end, the connecting region 34b of the second mounting part 30b comprises a truncated-cone-shaped wall portion 44, which tapers in the apical direction.

During the connection of the second mounting part 30b to the dental implant 10, the connecting region 34b is introduced axially into the receiver 16, the corresponding cylindrical wall portions 20 and 36b and the corresponding anti-rotation devices 22 and 38b engaging one another in the same manner as in the first mounting part 30a. The introduction depth of the connecting portion 34b is limited in this case by the shape-locking and optionally frictional mutual engagement of the corresponding truncated-cone-shaped wall portions 18 and 44, the perpendicular support face 16 of the dental implant 10 not coming into contact with a corresponding mating face. The truncated-cone-shaped wall portions 18 and 44 are therefore used for the axial and radial support of the second mounting part 30b, and the corresponding cylindrical wall portions 20 and 36b are used for the additional radial support.

The dental implant system according to the invention according to FIGS. 1 to 5 can additionally comprise further first and second mounting parts, the connecting region of which is configured identically to in the mounting parts 30a or 30b, which, however, instead of the carrier region 32a or 32b, have a different design, in other words are not configured as abutments, but, for example, as impression posts or gingiva formers. In a system of this type, the dentist can decide individually for each treatment step of an implantology treatment whether he will use a first mounting part with a connecting region 34a for a cylindrical connection or a second mounting part with a connecting region 34b for a conical connection, depending on whether as precise an axial positioning of the mounting part as possible or as good a transmission of force as possible from the mounting part to the dental implant is paramount.

### LIST OF REFERENCE NUMERALS

10 dental implant
12 external thread
14 transverse groove
16 receiver
18 truncated-cone-shaped wall portion
20 cylindrical wall portion
22 anti-rotation device
24 recess
26 perpendicular support face
30a first dental mounting part
32a carrier region
34a connecting region
36a cylindrical wall portion
38a anti-rotation device
40a projection
42 perpendicular support face
30b second dental mounting part
32b carrier region
34b connecting region
36b cylindrical wall portion
38b anti-rotation device
40b projection
44 truncated-cone-shaped wall portion

1. A dental implant with a receiver for dental mounting parts, wherein the receiver comprises an apical end and a coronal end and extends along a longitudinal axis of the dental implant and a dental mounting part can be introduced axially into the receiver, wherein the receiver comprises at least one cylindrical wall portion and the dental implant has a support face perpendicular to the longitudinal axis, so that a corresponding first mounting part can be supported radially on the at least one cylindrical wall portion and axially on the support face, and in that the receiver furthermore comprises at least one truncated-cone-shaped wall portion, on which a corresponding second mounting part can be supported radially and axially.

2. The dental implant according to claim 1, wherein the cylindrical and truncated-cone-shaped wall portions are in each case configured rotationally symmetrically in relation to the longitudinal axis and are arranged consecutively in the axial direction.

3. The dental implant according to claim 1, wherein the receiver comprises a cylindrical wall portion in the region of its apical end and a truncated-cone-shaped wall portion in the region of its coronal end.

4. The dental implant according to as claim 1, wherein the perpendicular support face extends outwardly from the coronal end of the receiver.

5. The dental implant according to claim 1, wherein the perpendicular support face is formed by an apical base face of the receiver.

6. The dental implant according to claim 1, wherein the receiver comprises two cylindrical wall portions, between which a truncated-cone-shaped wall portion is arranged.

7. The dental implant according to claim 1, wherein the receiver comprises two truncated-cone-shaped wall portions, between which a cylindrical wall portion is arranged.

8. The dental implant according to claim 7, wherein the two truncated-cone-shaped wall portions lie on a same imaginary conical face.

9. The dental implant according to claim 1, wherein the at least one truncated-cone-shaped wall portion has an angle of inclination with respect to the longitudinal axis in the range from about 5° to about 15°.

10. The dental implant according to claim 1, wherein the receiver comprises a portion serving as an anti-rotation device, with which a corresponding portion of a dental mounting part can engage.

11. The dental implant according to claim 10, wherein the portion serving as an anti-rotation device is arranged between a cylindrical and a truncated-cone-shaped wall portion.

12. The dental implant according to claim 1, wherein the dental implant comprises a threaded bore, which apically adjoins the receiver and into which a screw can be screwed to fix a mounting part.

13. The dental implant according to claim 1, wherein the dental implant is a bone screw with an external thread for screwing into a jawbone.

14. The dental implant according to claim 1, wherein the dental implant is completely or partially formed from titanium, a titanium alloy, a cobalt-chromium alloy or ceramic.

15. A dental implant system, comprising the dental implant according to claim 1, and at least one first and at least one second dental mounting part, which can selectively be connected to the dental implant, the mounting parts in each case comprising a connecting region, which can be axially introduced into the receiver of the dental implant, wherein
the connecting region of the at least one first mounting part comprises at least one cylindrical wall portion and a support face perpendicular to the longitudinal axis, so that the corresponding cylindrical wall portions of the receiver and of the connecting region engage one another in shape-locking manner and the corresponding support faces abut one another when the mounting part is connected to the dental implant; and
the connecting region of the at least one second mounting part comprises at least one truncated-cone-shaped wall portion, so that the corresponding truncated-cone-shaped wall portions of the receiver and of the connecting region engage one another in shape-locking manner when the mounting part is connected to the dental implant.

16. The dental implant system according to claim 15, wherein the connecting portion of the at least one second mounting part additionally comprises a cylindrical wall portion mutually engaging with the corresponding cylindrical wall portion of the receiver in shape-locking manner when the mounting part is connected to the dental implant.

17. The dental implant system according to claim 15, wherein the connecting region of the at least one first mounting part comprises two cylindrical wall portions and/or wherein the connecting region of the at least one second mounting part comprises two truncated-cone-shaped wall portions.

18. The dental implant system according to claim 15, wherein the connecting region of the at least one first mounting part and/or of the at least one second mounting part in each case comprises a portion serving as an anti-rotation device and mutually engaging with a corresponding portion of the receiver of the dental implant in shape-locking manner when the first or the second mounting part is connected to the dental implant.
